(19) United States
(12) Patent Application Publication
(10) Pub. No.: US 2005/0288272 A1
(43) Pub. Date: Dec. 29, 2005

(54) Pharmaceutical compositions comprising NEP-inhibitors, inhibitors of the endogenous endothelin producing system and AT1 receptor antagonists

(75) Inventors: Dieter Ziegler, Hemmingen (DE); Klaus Witte, Hannover (DE); Matthias Straub, Barsinghausen (DE); Yvan Fischer, Barsinghausen (DE); Dirk Thormaehlen, Ronnenberg (DE); Dagmar Hoeltje, (US)

Correspondence Address: CROWELL & MORING LLP;INTELLECTUAL PROPERTY GROUP, P.O. BOX 14300, WASHINGTON, DC, 20044-4300, US

(73) Assignee: SOLVAY PHARMACEUTICALS GmbH, Hannover (DE)

(21) Appl. No.: 11/155,782

(22) Filed: Jun. 20, 2005

Related U.S. Application Data
(60) Provisional application No. 60/581,723, filed on Jun. 23, 2004.

(51) Int. Cl. A61K 31/55

(52) U.S. Cl. 514/212.07

(57) ABSTRACT

A combination therapy for cardiovascular diseases, in particular essential hypertension, pulmonary hypertension and/or congestive heart failure, involving administering a synergistic combination of at least one inhibitor of neutral endopeptidase, at least one inhibitor of the endogenous endothelin producing system, and at least one AT₁ receptor antagonist.

### CROSS REFERENCE TO RELATED APPLICATIONS

This application claims priority from U.S. provisional patent application No. 60/581,723, filed Jun. 23, 2005, the entire disclosure of which is incorporated herein by reference.

### BACKGROUND OF THE INVENTION

The present invention relates to a novel combination therapy for cardiovascular diseases, in particular essential hypertension, pulmonary hypertension and/or congestive heart failure, using a synergistic combination of at least one inhibitor of neutral endopeptidase (=NEP), at least one inhibitor of the endogenous endothelin producing system and at least one AT₁ receptor antagonist. The invention also thus relates to novel pharmaceutical compositions comprising NEP inhibitors, inhibitors of the endogenous endothelin producing system and AT₁ receptor antagonists and the use of said pharmaceutical composition in the inhibition or treatment of cardiovascular diseases in humans and other mammals.

The nature of cardiovascular, in particular hypertensive vascular, diseases is multifactorial. Combination therapy has been shown to address the multiple pathophysiologic factors that play a role in blood pressure elevation, including blood volume, vasoconstriction, and the impact of sympathetic nervous system and Renin-Angiotensin-Aldosterone-System (=RAAS) activity (see e.g. M. R. Weir, American Journal of Hypertension 11 (1998) 163S-169S), potentially resulting in both greater reduction in blood pressure and in lowered risks for target-organ damage. The use of a fixed, low-dose combination agent could also offer lower doses of each component than those that may be necessary with monotherapy, thus reducing the risks of dose-dependent adverse events and associated compliance problems.

It is known from U.S. Pat. No. 4,749,688 (=EP 254,032) that NEP inhibitors can lower blood pressure under conditions where angiotensin converting enzyme (=ACE) inhibitors as a monotherapy are relatively ineffective. Further, this document discloses that NEP inhibitors may be combined with other drugs used in the treatment of hypertension, e.g. ACE inhibitors, to enhance the effects of those drugs. Consequently, pharmaceutical compositions comprising both a NEP inhibitor and an ACE inhibitor are described.

Published US patent application no. U.S. 2003/0144215 (=WO 03/059345) discloses pharmaceutical compositions comprising a specific AT₁ receptor antagonist, valsartan, and NEP inhibitors for the treatment or inhibition of inter alia cardiovascular diseases.

Although the beneficial role of NEP inhibiting compounds in the treatment or inhibition of cardiovascular diseases, in particular essential hypertension, pulmonary hypertension and/or congestive heart failure, is widely acknowledged today, their profile of action is still suffering from certain inherent deficiencies. In congestive heart failure, as a result of the decreased cardiac output and the increase in peripheral resistance, back-pressure phenomena of the blood occur in the pulmonary circulation and the heart itself. As a result, an increased wall tension of the heart muscle occurs in the area of the auricles and chambers. In such a situation, the heart functions as an endocrine organ and secretes, inter alia, the atrial natriuretic peptide (=ANP) into the bloodstream. Due to its marked vasodilatory and natriuretic/diuretic activity, ANP brings about both a reduction in the peripheral resistance and a decrease in the circulating blood volume. The consequence is a marked pre- and afterload decrease. This constitutes an endogenous cardioprotective mechanism. This positive endogenous mechanism is limited in that ANP has only a very short half-life in the plasma. The reason for this is that the hormone is very rapidly broken down by NEP. Therefore, pharmacological NEP inhibition increases ANP levels and thus promotes this cardioprotective mechanism.

In congestive heart failure, due to a disease-related reduced output of the heart, a reflex increase in peripheral vascular resistance occurs. As a result, the heart muscle must begin to pump against an increased afterload. In a vicious cycle, this results in increased strain on the heart and worsens the situation further. The increase in the peripheral resistance is mediated, inter alia, by the vasoactive peptide endothelin. Endothelin (=ET) is the strongest presently known endogenous vasoconstrictory substance and is formed from the precursor big endothelin (=bigET) with participation of the endothelin converting enzyme (=ECE). NEP is involved not only in the breakdown of ANP but also in the breakdown of endothelin.

For these reasons, a combination of compounds having NEP-inhibiting activity with compounds capable of inhibiting the endogenous endothelin producing system or compounds with dual inhibiting activities on NEP and the endogenous endothelin producing system would seem to provide added value in the therapy of cardiovascular diseases like essential hypertension, pulmonary hypertension and/or congestive heart failure. As a result of inhibition of the endogenous endothelin producing system, formation of endothelin would be inhibited and thus an increase in peripheral resistance would be counteracted, which consequently leads to a relief of the strain on the heart muscle. As a result of inhibition of the ANP degrading enzyme NEP, higher ANP levels and an increased duration of action of ANP can be achieved. This will lead to a reinforcement of the ANP-mediated endogenous cardioprotective mechanism of action. However, because NEP may also be involved in ET degradation, a pure NEP inhibition would, in addition to the desired increase in the ANP levels, also lead to an unfavorable increase in the ET levels. For this reason, a mixed profile with dually acting inhibition of NEP and of the endogenous endothelin producing system is to be regarded as particularly favorable, since it prevents both the breakdown of the natriuretically/diuretically acting ANP (by NEP-blockade), and simultaneously inhibits the formation of ET. As a result, the adverse attendant effect of pure NEP-inhibitors (increase in the endothelin levels) no longer comes to bear.

Compounds with a dually acting combined inhibitory effect on NEP and the endogenous endothelin producing system, i.e. benzazepine-, benzoxazepine- and benzothiazepine-N-acetic acid derivatives, are known from U.S. Pat. No. 5,677,297 (=EP 733,642). Further advantageous pharmacological properties of compounds falling within the structural scope of U.S. Pat. No. 5,677,297 are known from U.S. Pat. No. 5,783,573 (=EP 830,863), U.S. Pat. No. 6,482,820 (=WO 00/48601) and US published application no. U.S. 2003/0040512 (=WO 01/03699).

Phosphonic acid substituted benzazepinone-N-acidic acid derivatives with a combined inhibitory effect on NEP and the endogenous endothelin producing system are disclosed in U.S. Pat. No. 5,952,327 (=EP 916,679).

Amidomethyl-substituted 1-(carboxyalkyl)- cyclopentylcarbonylaminobenzazepine-N-acetic acid derivatives which are useful e.g. for the treatment and/or inhibition of cardiovascular conditions or diseases, are disclosed in published international application no. WO 2005/030795.

It is known from published US patent application no. U.S. 2004/0162345 (=WO 02/094176) that certain compounds, including those disclosed in U.S. Pat. Nos. 5,677,297 and 5,952,327, may inhibit the endogenous endothelin producing system via an inhibition of metalloprotease IGS5. The metalloprotease IGS5 is also known as human soluble endopeptidase (=hSEP) and is described e.g. in U.S. 2004/0162345. Further, U.S. 2004/0162345 discloses the use of compounds with combined NEP/hSEP inhibitory activity for the inhibition or treatment of inter alia cardiovascular diseases.

### SUMMARY OF THE INVENTION

It is the object of the present invention to provide a novel combination therapy for cardiovascular diseases, in particular essential hypertension, pulmonary hypertension and/or congestive heart failure, with enhanced efficacy and an advantageous safety profile.

It has now surprisingly been found that a combination of at least one NEP-inhibitor, at least one inhibitor of the endogenous endothelin producing system and additionally at least one AT₁ receptor antagonist, provides still further enhanced efficacy in cardiovascular diseases like essential hypertension, pulmonary hypertension and/or congestive heart failure, and a good safety profile.

The invention therefore relates in a first aspect to pharmaceutical compositions comprising pharmacologically effective quantities of each of
a) at least one NEP-inhibitor as a first active agent,
b) at least one inhibitor of the endogenous endothelin producing system as a second active agent, and
c) at least one AT₁ receptor antagonist as a third active agent.
   The pharmaceutical compositions according to the invention may further and preferably comprise conventional pharmaceutically acceptable auxiliaries and/or carriers. The pharmaceutical compositions according to the invention may further comprise acetylsalicylic acid.

Inhibitors of the endogenous endothelin producing system can be selected from the group consisting of inhibitors of ECE, inhibitors of hSEP and dually acting compounds capable of inhibiting ECE and hSEP. Dually acting compounds capable of inhibiting ECE and hSEP are preferred.

In the pharmaceutical compositions according to the invention, the subcombination of at least one NEP-inhibitor (a) and at least one inhibitor of the endogenous endothelin producing system (b) can preferably be realized by a single dually acting compound capable of inhibiting NEP and the endogenous endothelin producing system. Dually acting compounds capable of inhibiting both NEP and hSEP are preferred. Particularly preferred are the dually acting compounds of general Formula I, 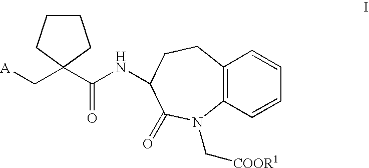
wherein
R¹ is hydrogen or a group forming a biolabile carboxylic acid ester
A represents a group selected from the subgroups
   a, 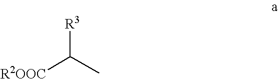
      wherein
   R² is hydrogen or a a group forming a biolabile carboxylic acid ester and
   R³ is a phenyl-C₁₋₄-alkyl group which can optionally be substituted in the phenyl ring by C₁₋₄-alkyl, C₁₋₄-alkoxy or halogen; or a naphthyl-C₁₋₄-alkyl group; or
   b, 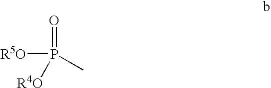
      wherein
   R⁴ is hydrogen or a group forming a biolabile phosphonic acid ester and
   R⁵ is hydrogen or a group forming a biolabile phosphonic acid ester; or
   c, 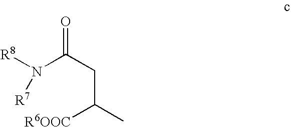
      wherein
   R⁶ is is hydrogen or a group forming a biolabile carboxylic acid ester,
   R⁷ is hydrogen, C₁₋₄-alkyl or C₁₋₄-hydroxyalkyl, the hydroxyl group of which is optionally esterified with C₂₋₄-alkanoyl or an amino acid residue, and
   R⁸ is C₁₋₄-alkyl; C₁₋₄-alkoxy-C₁₋₄-alkyl; C₁₋₄-hydroxyalkyl, which is optionally substituted by a second hydroxyl group and the hydroxyl groups of which are each optionally esterified with C₂₋₄-alkanoyl or an amino acid residue; (C₀₋₄-alkyl)₂amino-C₁₋₆-alkyl; C₃₋₇-cycloalkyl; C₃₋₇-cycloalkyl-C₁₋₄-alkyl; phenyl-C₁₋₄-alkyl, the phenyl group of which is optionally substituted 1-2 times by C₁₋₄-alkyl, C₁₋₄-alkoxy and/or halogen; naphthyl-C₁₋₄-alkyl; C₃₋₆-oxoalkyl; phenylcarbonylmethyl, the phenyl group of which is optionally substituted 1-2 times by C₁₋₄-alkyl, C₁₋₄-alkoxy and/or halogen, or 2-oxoazepanyl, or
   R⁷ and R⁸ together are C₄₋₇-alkylene, the methylene groups of which are optionally replaced 1-2 times by carbonyl, nitrogen, oxygen and/or sulfur and which are optionally substituted once by hydroxy, which is optionally esterified with C₂₋₄-alkanoyl or an amino acid residue; C₁₋₄-alkyl; C₁₋₄-hydroxyalkyl, the hydroxyl group of which is optionally esterified with C₂₋₄-alkanoyl or an amino acid residue; phenyl or benzyl,
      and/or physiologically compatible salts of acids of Formula I and/or physiologically compatible acid addition salts of compounds of Formula Ic.

Where the substituents in the compounds of Formula I are or contain C₁₋₄-alkyl groups, these may be straight-chain or branched. Where biolabile ester forming groups in the compounds of Formula I are or contain lower alkyl groups, these may be straight-chain or branched and usually contain 1 to 4 carbon atoms. Where the substituents contain halogen, fluorine, chlorine or bromine, preferably fluorine or chlorine, are particularly suitable. Where substituents contain C₂₋₄-alkanoyl, this may be straight-chain or branched. Acetyl is preferred as C₂₋₄-alkanoyl.

Where substituents are biolabile ester forming groups, these as a rule represent prodrugs of the active drug principle. Prodrugs are therapeutic agents which are inactive per se but are transformed into one or more active metabolites. Prodrugs are bioreversible derivatives of drug molecules used to overcome some barriers to the utility of the parent drug molecule. These barriers include, but are not limited to, solubility, permeability, stability, presystemic metabolism and targeting limitations (see e.g. Medicinal Chemistry: Principles and Practice, 1994, ISBN 0-85186-494-5, Ed.: F. D. King, p. 215; J. Stella, “Prodrugs as therapeutics”, Expert Opin. Ther. Patents, 14(3), 277-280, 2004; P. Ettmayer et al., “Lessons learned from marketed and investigational prodrugs”, J. Med. Chem., 47, 2393-2404, 2004).

Suitable physiologically compatible salts of free acids or partial esters of Formula I include their alkali metal, alkaline earth metal or ammonium salts, for example sodium or calcium salts or salts with physiologically compatible, pharmacologically neutral organic amines such as, for example, diethylamine or tert.-butylamine.

Preferred are the compounds of general Formula Ia, 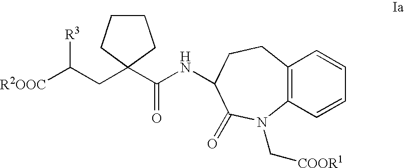
wherein R¹, R² and R³ have the above meanings, and physiologically compatible salts of acids of Formula Ia. Preferred salts of compounds of Formula Ia are e.g. disclosed in document WO 03/059939 A1 which is incorporated herein by reference. The compounds of Formula Ia contain two chiral carbon atoms, namely the carbon atom which is in the 3 position of the ring framework (=3-position) and bears the amide side-chain, and the carbon atom of the amide side-chain which bears the radical R³ (=2′-position). The compounds can therefore exist in several optically active stereoisomeric forms or as a racemate. According to the present invention both the racemic mixtures and the isomerically pure compounds of Formula Ia may be used.

The compounds of Formula Ia are optionally esterified dicarboxylic acid derivatives. Depending on the form of administration, biolabile monoesters, particularly compounds in which R² is a group forming a biolabile ester and R¹ is hydrogen, or dicarboxylic acids are preferred, the latter being particularly suitable for i.v. administration. Groups which can be cleaved under physiological conditions in vivo, releasing bioavailable derivatives of the compounds of Formula Ia, are suitable as groups forming biolabile carboxylic acid esters R¹ and R². Suitable examples of this are C₁₋₄-alkyl groups, in particular methyl, ethyl, n-propyl and isopropyl; C₁₋₄-alkyloxy-C₁₋₄-alkyloxy-C₁₋₄-alkyl groups, in particular methoxyethoxymethyl; C₃₋₇-cycloalkyl groups, in particular cyclohexyl; C₃₋₇ cycloalkyl-C₁₋₄-alkyl groups, in particular cyclopropylmethyl; N,N-di-(C₀₋₄-alkyl)amino-C₁₋₆-alkyl groups; phenyl or phenyl-C₁₋₄-alkyl groups optionally substituted in the phenyl ring once or twice by halogen, C₁₋₄-alkyl or C₁₋₄-alkoxy or by a C₁₋₄-alkylene chain bonded to two adjacent carbon atoms; dioxolanylmethyl groups optionally substituted in the dioxolane ring by C₁₋₄-alkyl; C₂₋₆-alkanoyloxy-C₁₋₄-alkyl groups optionally substituted at the oxy-C₁₋₄-alkyl group by C₁₋₄-alkyl; double esters like 1-[[(C₁₋₄-alkyl)carbonyl]oxy]C₁₋₄-alkyl esters, e.g. (RS)-1-[[(isopropyl)carbonyl]oxy]ethyl or (RS)-1-[[(ethyl)carbonyl]oxy]-2-methylpropyl (for preparation see e.g. F. W. Sum et al., Bioorg. Med. Chem. Lett. 9 (1999) 1921-1926 or Y. Yoshimura et al., The Journal of Antibiotics 39/9 (1986) 1329-1342); carbonate esters like 1-[[(C₄₋₇-cycloalkyloxy)carbonyl]oxy] C₁₋₄-alkyl esters, preferably (RS)-1-[[(cyclohexyloxy)carbonyl]oxy]ethyl (=cilexetil; for preparation see e.g. K. Kubo et al., J. Med. Chem. 36 (1993) 2343-2349, cited as “Kubo et al.” hereinafter)) or 2-oxo-1,3-dioxolan-4-yl—C₁₋₄-alkyl esters which optionally contain a double bond in the dioxolan ring, preferably 5-methyl-2-oxo-1,3-dioxolen-4-yl-methyl (=medoxomil, for preparation see e.g. Kubo et al.) or 2-oxo-1,3-dioxolan-4-yl-methyl (=(methyl)ethylenecarbonate). Where the group forming a biolabile ester represents an optionally substituted phenyl-C₁₋₄-alkyl group, this may contain an alkylene chain with 1 to 3, preferably 1, carbon atoms and preferably stands for optionally substituted benzyl, in particular for 2-chlorobenzyl or 4-chlorobenzyl. Where the group forming a biolabile ester represents an optionally substituted phenyl group, the phenyl ring of which is substituted by a lower alkylene chain, this may contain 3 to 4, preferably 3, carbon atoms and in particular be indanyl. Where the group forming a biolabile ester represents an optionally substituted C₂₋₆-alkanoyloxy-C₁₋₄-alkyl group, the C₂₋₆-alkanoyl group may be straight-chain or branched.

R¹ preferably has the meanings hydrogen, C₁₋₄-alkyl, p-methoxybenzyl, N,N-di-(C₀₋₄-alkyl)amino-C₁₋₆-alkyl, (RS)-1-[[(isopropyl)carbonyl]oxy]ethyl, (RS)-1-[[(ethyl) carbonyl]oxy]-2-methyl propyl, (RS)- 1-[[(cyclohexyloxy)carbonyl]oxy]ethyl, 5-methyl-2-oxo-1,3-dioxolen-4-yl-methyl, 2-oxo-1,3-dioxolan-4-yl-methyl or (RS)- 1-[[(ethoxy)carbonyl]oxy]ethyl.

R² preferably has the meanings hydrogen, ethyl, methoxyethoxymethyl, (RS)-1-[[(isopropyl)carbonyl]oxy]ethyl, (RS)-1-[[(ethyl)carbonyl]oxy]-2-methylpropyl, (RS)-1-[[(cyclohexyloxy)carbonyl]oxy]ethyl, 5-methyl-2-oxo-1,3-dioxolen-4-yl-methyl, 2-oxo-1,3-dioxolan-4-yl-methyl or (RS)-1-[[(ethoxy)carbonyl]oxy]ethyl.

More preferred are the compounds which are selected from the group consisting of 2-[1-(1-carboxymethyl-2-oxo-2,3,4,5-tetrahydro-1H-benzo[b]azepin-3-ylcarbamoyl)-cyclopentylmethyl]-4-phenyl-butyric acid ethyl ester [alternative name: 3-[1-{2′-(ethoxycarbonyl)}-4′-phenylbutyl]-cyclopentan-1-carbonylamino]-2,3,4,5-tetrahydro-2-oxo-1H-1-benzazepin-1-acetic acid] of Formula II, 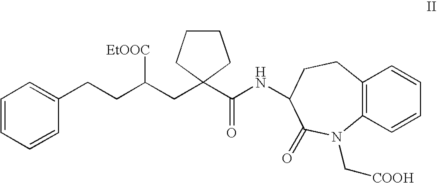
2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid ethyl ester [alternative name: 3-[1-{2-(ethoxycarbonyl)-4-(1-naphthyl)butyl]cyclopentyl}carbonyl)amino]-2-oxo-2,3,4,5-tetrahydro-1H-1-benzazepin-1-yl}acetic acid] of Formula III, 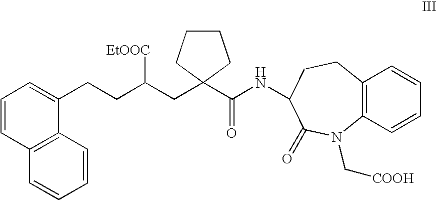
2-[1-(1-carboxymethyl-2-oxo-2,3,4,5-tetrahydro-1H-benzo[b]azepin-3-ylcarbamoyl)-cyclopentylmethyl]-4-phenyl-butyric acid of Formula IV, 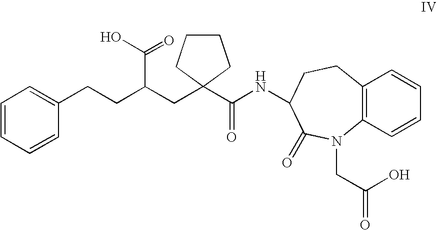
2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid of Formula V, 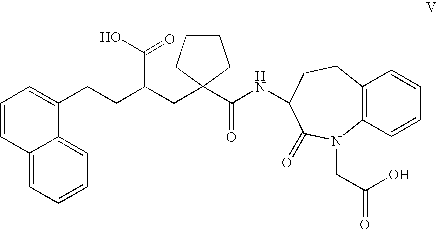
and physiologically compatible salts of the acids of Formulas II, III, IV and/or V. The compounds of Formulas II, III, IV and V are especially suited in their 3S,2′R forms. Most preferred is the compound of Formula II in its 3S,2′R form, also known as “daglutril” or “SLV306”. The compounds of Formula Ia are known, for example, from document EP 0 733 642 A1 which is incorporated herein by reference, and can be produced according to the production processes disclosed or referenced in this document or analogously to said production processes.

Further, compounds of general Formula Ib, 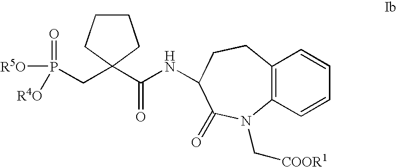
wherein R¹, R⁴ and R⁵ have the meanings given above, or physiologically compatible salts of acids of Formula Ib can be used as dually acting compounds capable of inhibiting NEP and the endogenous endothelin producing system. The compounds of Formula Ib are known, for example, from document EP 0 916 679 A1 which is incorporated herein by reference, and can be produced according to the production processes disclosed or referenced in this document or analogously to said production processes.

Suitable groups R¹ forming biolabile carboxylic acid esters in compounds of Formula Ib are those as specified for compounds of Formula Ia above.

Groups R⁴ and R⁵ suitable as groups forming biolabile phosphonic acid esters are those which can be removed under physiological conditions in vivo with release of the respective phosphonic acid function. For example, groups which are suitable for this purpose are lower alkyl groups, C₂-C₆-alkanoyloxymethyl groups optionally substituted on the oxymethyl group by lower alkyl, or phenyl or phenyl-lower alkyl groups whose phenyl ring is optionally mono- or polysubstituted by lower alkyl, lower alkoxy or by a lower alkylene chain bonded to two adjacent carbon atoms. If the group R⁴ and/or R⁵ forming a biolabile ester is or contains lower alkyl, this can be branched or unbranched and can contain 1 to 4 carbon atoms. If R⁴ and/or R⁵ are an optionally substituted alkanoyloxymethyl group, it can contain a preferably branched alkanoyloxy group having 2 to 6, preferably 3 to 5, carbon atoms and can, for example, be a pivaloyloxymethyl radical (=tert-butylcarbonyloxymethyl radical). If R⁴ and/or R⁵ are an optionally substituted phenyl-lower alkyl group, this can contain an alkylene chain having 1 to 3, preferably 1, carbon atoms. If the phenyl ring is substituted by a lower alkylene chain, this can contain 3 to 4, in particular 3, carbon atoms and the substituted phenyl ring is in particular indanyl.

The compounds of the formula Ib contain a chiral carbon atom, namely the carbon atom carrying the amide side chain in the 3-position of the benzazepine structure. The compounds can thus be present in two optically active stereoisomeric forms or as a racemate. The present invention includes both the racemic mixtures and the isomerically pure compounds of the formula I. If R⁴ and R⁵ in compounds of the formula Ib are not hydrogen and in each case have different meanings, the phosphorus atom of the phosphonic acid group can also be chiral. The invention also relates to the isomer mixtures and isomerically pure compounds of the formula Ib formed as a result of chiral phosphorus atoms.

When compounds of Formula Ib are used according to the invention, (3-{[1-(benzyloxy-ethoxy-phosphorylmethyl)-cyclopentanecarbonyl]-amino}-2-oxo-2,3,4,5-tetrahydro-benzo[b]azepin-1-yl)-acetic acid tert-butyl ester and isobutyric acid 1-[[1-(−1-carboxymethyl-2-oxo-2,3,4,5-tetrahydro-1H-benzo[b]azepin-3-ylcarbamoyl)- cyclopentylmethyl]-(1-isobutyryloxy-ethoxy)-phosphinoyloxy]-ethyl ester are preferred. Both of said compounds are particularly preferred when the stereochemistry at the chiral carbon atom (see above) is “S”, namely in their “(3S)” configuration. The compounds of Formula Ib are known, for example, from document EP 0 916 679 A1, and can be produced according to the production processes disclosed or referenced in this document or analogously to said production processes.

Also preferred are the compounds of general Formula Ic, 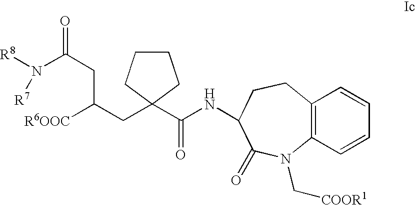
wherein R¹, R⁶, R⁷ and R⁸ have the above meanings, and physiologically compatible salts of acids of Formula Ic and/or physiologically compatible acid addition salts of compounds of Formula Ic, for the use as dually acting compounds capable of inhibiting NEP and the endogenous endothelin producing system in pharmacological compositions according to the invention. The compounds of Formula Ic are known, for example, from document WO 2005/030795 A1 which is incorporated herein by reference, and can be produced according to the production processes disclosed or referenced in this document or analogously to said production processes.

Where in compounds of Formula Ic the substituents R⁷ and/or R⁸ contain basic groups, in particular nitrogen, the compounds of Formula Ic may also occur in the form of acid addition salts. Physiologically compatible acid addition salts of compounds of Formula Ic are their conventional salts with inorganic acids, for example sulfuric acid, phosphoric acid or hydrohalic acids, preferably hydrochloric acid, or with organic acids, for example lower aliphatic monocarboxylic, dicarboxylic or tricarboxylic acids such as maleic acid, fumaric acid, tartaric acid, citric acid, or with sulfonic acids, for example lower alkanesulfonic acids such as methanesulfonic acid.

Suitable groups R¹ forming biolabile carboxylic acid esters in compounds of Formula Ic are those as specified for compounds of Formula Ia above. Suitable groups R⁶ forming biolabile carboxylic acid esters in compounds of Formula Ic are the same as specified for groups R² in compounds of Formula Ia above.

R⁷ preferably has the meanings hydrogen, methyl, ethyl, 2-hydroxyethyl or 3-hydroxypropyl, each hydroxyl group optionally being esterified with C₂₋₄-alkanoyl or an amino acid residue.

Where R⁸ has the meaning (C₀₋₄-alkyl)₂amino-C₁₋₆-alkyl, one or two C₀₋₄-alkyl groups can independently of each other be present. More specifically, “(C₀₋₄-alkyl)₂amino-C₁₋₆-alkyl” expressly comprises the meanings “(C₀)₂-alkylamino-C₁₋₆-alkyl”, “(C₀)(C₁₋₄)-alkylamino-C₁₋₆-alkyl” and “(C₁₋₄)₂-alkylamino-C₁₋₆-alkyl”. “(C₀)₂-alkylamino-C₁₋₆-alkyl” is meant to denominate an unsubstituted primary (=—NH₂) amino group bonded to C₁₋₆-alkyl(en); “(C₀)(C₁₋₄)-alkylamino-C₁₋₆-alkyl” is meant to denominate a secondary amino group monosubstituted by (C₁₋₄)-alkyl and bonded to C₁₋₆-alkyl(en); “(C₁₋₄)₂-alkylamino-C₁₋₆-alkyl” is meant to denominate a tertiary amino group disubstituted by (C₁₋₄)-alkyl and bonded to C₁₋₆-alkyl(en). R⁸ preferably has the meanings isopropyl; methoxyethyl; 2-hydroxyethyl or 3-hydroxypropyl, each hydroxyl group optionally being esterified with C₂₋₄-alkanoyl or an amino acid residue; 3-acetyloxy-n-propyl; cyclopropylmethyl; 2-methoxybenzyl, 4-methoxybenzyl; 4-methoxyphenylethyl; 2,4-dimethoxybenzyl; 1-naphthylmethyl; 3-oxo-1,1-dimethylbutyl; phenyl-2-oxoethyl; 2-(4-methoxyphenyl)-2-oxoethyl; 3-(2-oxoazepanyl); (C₀₋₄-alkyl)₂amino-C₁₋₆-alkyl, in particular dimethylamino-n-propyl, (methyl)aminoethyl, amino-n-propyl, amino-n-butyl or amino-n-pentyl.

Where R⁷ and R⁸ together are C₄₋₇-alkylene, the methylene groups of which are optionally replaced or optionally substituted, in each case morpholine; piperidine; 4-ketopiperidine; 4-hydroxypiperidine, optionally being esterified with C₂₋₄-alkanoyl or an amino acid residue at the hydroxyl group; piperazine or pyrrolidine is preferred.

Where in the compounds of Formula Ic hydroxyl groups are esterified with amino acid residues, these amino acid residues may be derived from natural or non-natural, α- or β-amino acids. Suitable amino acids which can be used are for example selected from the group cosisting of alanine, 2-aminohexanoic acid (=norleucine), 2-aminopentanoic acid (=norvaline), arginine, asparagine, aspartic acid, cysteine, 3,4-dihydroxyphenylalanine (=dopa), glutamine, glutamic acid, glycine, histidine, isoleucine, leucine, lysine, methionine, ornithine (=2,5-diaminovaleric acid), 5-oxo-2-pyrrolidinecarbonic acid (=pyroglutamic acid), phenylalanine, proline, serine, threonine, thyronine, tryptophan, tyrosine and valine. Preferred are amino acid residues which are derived from alanine, asparagine, glutamine, glycine, isoleucine, leucine, lysine, ornithine, phenylalanine, proline and valine.

The compounds of Formula Ic contain two chiral carbon atoms, namely the carbon atom bearing the amide side chain in position 3 of the benzazepine skeleton (=C_{b}*) and the carbon atom bearing the radical “—COOR⁶” (=Cₐ*). The compounds can thus be present in a total of four stereoisomeric forms. The present invention comprises both the mixtures of stereoisomers and enantiomers, and also the isomerically pure compounds of Formula Ic. Isomerically pure compounds of Formula Ic are preferred. Particularly preferred are compounds of Formula Ic wherein the carbon atom bearing the amide side chain in position 3 of the benzazepine skeleton is in the “S” configuration. With respect to the chiral carbon atom “*Cₐ” bearing the radical “—COOR⁶”, the configuration of the compounds of Formula I which is preferred according to the invention in the context of this invention is provisionally assigned the configuration designation “rel1”. It can be derived by analogous observations of suitable compounds of known configuration that the preferred configuration “rel1” at the chiral center “*Cₐ” is probably likewise the “S” configuration.

Particularly preferred compounds of Formula Ic are selected from the group consisting of:
2-{[1-({[1-(carboxymethyl)-2-oxo-2,3,4,5-tetrahydro-1H-1-benzazepin-3-yl]amino}-carbonyl)cyclopentyl]methyl}-4-[isopropyl(methyl)amino]-4-oxobutanoic acid;
2-{[1-({[1-(carboxymethyl)-2-oxo-2,3,4,5-tetrahydro-1H-1-benzazepin-3-yl]amino}carbonyl)cyclopentyl]methyl}-4-(dimethylamino)-4-oxobutanoic acid;
2-{[1-({[1-(carboxymethyl)-2-oxo-2,3,4,5-tetrahydro-1H-1-benzazepin-3-yl]amino}carbonyl)cyclopentyl]methyl}-4-(diethylamino)-4-oxobutanoic acid;
2-{[1-({[1-(carboxymethyl)-2-oxo-2,3,4,5-tetrahydro-1H-1-benzazepin-3-yl]amino}-carbonyl) cyclopentyl]methyl}-4-[(2-hydroxyethyl) (methyl) amino]-4-oxobutanoic acid;
2-{[1-({[1-(carboxymethyl)-2-oxo-2,3,4,5-tetrahydro-1H-1-benzazepin-3-yl]amino}-carbonyl) cyclopentyl]methyl}-4-[(3-hydroxypropyl)(methyl)amino]-4-oxobutanoic acid;
2-{[1-({[1-(carboxymethyl)-2-oxo-2,3,4,5-tetrahydro-1H-1-benzazepin-3-yl]amino}-carbonyl) cyclopentyl]methyl}-4-(4-hydroxypiperidin-1-yl)- 4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-oxo-4-[4-(L-valyloxy)piperidin-1-yl]butanoic acid;
2-{[1-({[1-(carboxymethyl)-2-oxo-2,3,4,5-tetrahydro-1H-1-benzazepin-3-yl]amino}-carbonyl)cyclopentyl]methyl}-4-morpholin-4-yl-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-oxo-4-(4-oxopiperidin-1-yl)butanoic acid;
4-[bis(2-hydroxyethyl) amino]-2-{[1-({[1-(carboxymethyl)- 2-oxo-2,3,4,5-tetra hydro-1H-1-benzazepin-3-yl]amino}carbonyl)cyclopentyl]methyl}-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-{ethyl[3-(ethylamino)propyl]amino}-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-[[2-(dimethylamino)ethyl](methyl)amino]-4-oxobutanoic acid;
4-[(3-aminopropyl) (ethyl)amino]-2-{[1-({[1-(carboxymethyl)- 2-oxo-2,3,4,5-tetrahydro-1H-1-benzazepin-3-yl]amino}carbonyl)cyclopentyl]methyl}-4-oxobutanoic acid,
2-{[1-({[1-(carboxymethyl)-2-oxo-2,3,4,5-tetrahydro-1H-1-benzazepin-3-yl]amino}-carbonyl)cyclopentyl]methyl}-4-{methyl[2-(methylamino)ethyl]amino}-4-oxobutanoic acid;
4-[(4-aminobutyl)(methyl)amino]-2-{[1-({[1-(carboxymethyl)-2-oxo-2,3,4,5-tetrahydro-1H-1-benzazepin-3-yl]amino}carbonyl)cyclopentyl]methyl}-4-oxobutanoic acid;
4-[(4-aminobutyl) (ethyl)amino]-2-{[1-({[1-(carboxymethyl)- 2-oxo-2,3,4,5-tetrahydro-1H-1-benzazepin-3-yl]amino}carbonyl) cyclopentyl]methyl}-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-{methyl[3-(methylamino)propyl]amino}-4-oxobutanoic acid, and
4-[(5-aminopentyl)(methyl)amino]-2-{[1-({[1-(carboxymethyl)- 2-oxo-2,3,4,5-tetrahydro-1H-1-benzazepin-3-yl]amino}carbonyl)cyclopentyl]methyl}-4-oxobutanoic acid,
   together with their biolabile esters and physiologically compatible salts of acids of these compounds of Formula Ic and/or physiologically compatible acid addition salts of these compounds of Formula Ic.

AT₁ receptor antagonists are pharmacologically active drug compounds which are capable to selectively block the AT₁ subtype of the angiotensin II receptor in mammals and humans and which are known to possess e.g. antihypertensive properties. AT₁ receptor antagonists which can be used according to the present invention may be selected from the group consisting of abitesartan, benzyllosartan, candesartan, elisartan, embusartan, enoltasosartan, eprosartan, fonsartan, forasartan, glycyllosartan, irbesartan, isoteoline, losartan, milfasartan, olmesartan, opomisartan, pratosartan, ripisartan, saprisartan, saralasin, sarmesin, tasosartan, telmisartan, valsartan, zolasartan; Kissei KRH-94, Lusofarmaco LR-B/057, Lusofarmaco LR-B/081, Lusofarmaco LR B/087, Searle SC-52458, Sankyo CS-866, Takeda TAK-536, Uriach UR-7247, A-81282, A-81988, BIBR-363, BIBS39, BIBS-222, BMS-180560, BMS-184698, CGP-38560A, CGP-48369, CGP-49870, CGP-63170, CI-996, CV-11194, DA-2079, DE-3489, DMP-811, DuP-167, DuP-532, GA-0056, E-4177, EMD-66397, EMD-73495, EXP-063, EXP-929, EXP-3174, EXP-6155, EXP-6803, EXP-7711, EXP-9270, FK-739, HN-65021, HR-720, ICI-D6888, ICI-D7155, ICI-D8731, KR1-1177, KT3-671, KW-3433, L-158809, L-158978, L-159282, L-159689, L-159874, L-161177, L-162154, L-162234, L-162441, L-163007, L-163017, LY-235656, LY-285434, LY-301875, LY-302289, LY-315995, ME-3221, PD-123177, PD-123319, PD-150304, RG-13647, RWJ-38970, RWJ-46458, S-8307, S-8308, SL-91.0102, U-96849, U-97018, UP-269-6, UP-275-22, WAY-126227, WK-1492.2K, WK-1360, X-6803, XH-148, XR-510, YM-358, YM-31472, ZD-6888, ZD-7155 and ZD-8731 which are all known per se, or any physiologically compatible salts, solvates, prodrugs or esters thereof.

Preferred AT₁ receptor antagonists are selected from the group consisting of abitesartan, benzyllosartan, candesartan, elisartan, embusartan, enoltasosartan, eprosartan, fonsartan, forasartan, glycyllosartan, irbesartan, losartan, milfasartan, olmesartan, opomisartan, pratosartan, ripisartan, saprisartan, tasosartan, telmisartan, valsartan, zolasartan; Kissei KRH-94, Lusofarmaco LR-B/081, Searle SC-52458, Sankyo CS-866, Takeda TAK-536, Uriach UR-7247 or any physiologically compatible salts, solvates, prodrugs or esters thereof. Candesartan, eprosartan and losartan are more preferred AT₁ receptor antagonists. Eprosartan is usually used in the form of its mesylate. Losartan is usually used in the form of losartan potassium. Candesartan is usually used in the form of candesartan cilexetil.

Further pharmaceutical compositions which can be advantageously used in the treatment and/or inhibition of cardiovascular conditions or diseases comprise pharmacologically effective quantities of each of
a) at least one NEP-inhibitor as a first active agent,
b) at least one inhibitor of the endogenous endothelin producing system as a second active agent, and
d) at least one classic cardiovascular drug as a third or further active agent.

Suitable classic cardiovascular drugs can be selected from the group consisting of non-selective alpha-adrenoceptor antagonists, e.g. tolazoline or phenoxybenzamine; selective alpha-adrenoceptor antagonists, e.g. doxazosin, prazosin, terazosin or urapidil; beta-adrenoceptor antagonists, e.g. acebutolol, alprenolol, atenolol, betaxolol, bisoprolol, bupranolol, carazolol, carteolol, celiprolol, mepindolol, metipranolol, metoprolol, nadolol, oxprenolol, penbutolol, pindolol, propranolol, sotalol and timolol; mixed antagonists of alpha- and beta-adrenoceptors, e.g. carvedilol or labetolol; ganglion blockers, e.g. reserpine or guanethidine; alpha2-adrenoceptor agonists (including centrally acting alpha2-adrenoceptor agonists), e.g. clonidine, guanfacine, guanabenz methyldopa and moxonidine; renin-inhbitors, e.g. alskiren; ACE-inhbitors, e.g. benazepril, captopril, cilazapril, enalapril, fosinopril, imidapril, lisinopril, moexipril, quinapril, perindopril, ramipril, spirapril or trandolapril; mixed or selective endothelin receptor antagonists e.g. atrasentan, bosentan, clazosentan, darusentan, sitaxsentan, tezosentan, BMS-193884 or J-104132; direct vasodilators, e.g. diazoxide, dihydralazine, hydralazine or minoxidil; mixed ACE/NEP-inhbitors, e.g. omapatrilat; ECE-inhbitors, e.g. FR-901533; PD-069185; CGS-26303; CGS-34043; CGS-35066; CGS-30084; CGS-35066; SM-19712; Ro0677447; selective NEP-inhibitors; vasopressin antagonists, aldosterone receptor antagonists, e.g. eplerenone; angiotensin vaccine; and urotensin II receptor antagonists. Preferably, the classic cardiovascular drugs may be administered together with a drug selected from the group consisting of 2-[1-(1-Carboxymethyl-2-oxo-2,3,4,5-tetrahydro-1H-benzo[b]azepin-3-ylcarbamoyl)-cyclopentylmethyl]-4-phenyl-butyric acid ethyl ester; 2-[1-(1-Carboxymethyl-2-oxo-2,3,4,5-tetrahydro-1H-benzo[b]azepin-3-ylcarbamoyl)-cyclopentylmethyl]-4-naphthalen-1-yl-butyric acid ethyl ester; 2-[1-(1-Carboxymethyl-2-oxo-2,3,4,5-tetrahydro-1H-benzo[b]azepin-3-ylcarbamoyl)- cyclopentylmethyl]-4-phenyl-butyric acid; 2-[1-(1-carboxymethyl-2-oxo-2,3,4,5-tetrahydro-1H-benzo[b]azepin-3-ylcarbamoyl)-cyclopentyl methyl]-4-naphthalen-1-yl-butyric acid; and their physiologically compatible salts. More preferred, the classic cardiovascular drugs may be administered together with daglutril.

The pharmaceutical compositions according to the invention can be prepared in a manner known per se and thus can be obtained as formulations suitable for enteral, such as oral or rectal, or parenteral administration to mammals or humans, comprising a therapeutical effective amount of the pharmacologically active agents, alone or in combination with one or more pharmaceutically acceptable auxiliaries and/or carriers, especially suitable for enteral or parenteral application. Pharmaceutical compositions for enteral or parenteral administration are, for example, in unit dosage forms, such as coated tablets, tablets, capsules or suppositories and also ampoules. These are prepared in a manner which is known per se, for example using conventional mixing, granulation, coating, solubulizing or lyophilizing processes. Typical oral formulations include coated tablets, tablets, capsules, syrups, elixirs and suspensions. Capsules may contain the active agents e.g. in form of powders, granules, pellets, beadlets or microtablets. For example, a pharmaceutical composition according to the invention may consist of from about 0.1% to 90%, preferably of from about 1% to about 80%, of the active agents, the rest being made up by pharmaceutically acceptable auxiliaries and/or carriers. Thus, pharmaceutical compositions for oral use can be obtained by combining the active compounds with solid excipients, if desired granulating a mixture which has been obtained, and, if required or necessary, processing the mixture or granulate into tablets or coated tablet cores after having added suitable auxiliary substances. Typical injectable formulations include solutions and suspensions.

In one embodiment of the pharmaceutical compositions according to the invention, the active agents (a), (b) and (c) can be obtained and administered together, e.g. in one combined unit dosage form like in one tablet or capsule, i.e. in a physical combination. In such a combined unit dosage form, the different active agents (a), (b) and (c) can be segregated from each other, e.g. by using different layers in the tablet, e.g. by the use of inert intermediate layers known in the art; or by using different compartments in the capsule. The corresponding active agents or their pharmaceutically acceptable salts may also be used in form of their hydrates or include other solvents used for crystallization. A unit dosage form may be a fixed combination. A unit dosage form, in particular a fixed combination of the active agents (a), (b) and (c) is a preferred alternative of this embodiment. Fixed combinations comprising daglutril and eprosartan, daglutril and candesartan or daglutril and losartan are preferred embodiments of the invention.

In another embodiment the active agents (a), (b) and (c) can be obtained and administered in two or more separate unit dosage forms, e.g. in two or more tablets or capsules, the tablets or capsules being physically segregated from each other. The two or more separate unit dosage forms can be administered simultaneously or stepwise (separately), e.g. sequentially one after the other in either order. Thus, the active agents can be administered in either order at the same time or at different times spread over the day, the optimal dosage regimen usually being determined by prescription of a physician. When a dually acting compound capable of inhibiting NEP and the endogenous endothelin producing system is used to embody the combination of active agents (a) and (b), the active agents [(a)+(b)] and (c) in the pharmaceutical composition may advantageously be present in two separate dosage forms, usually complementary or balanced for combined use, e.g. as two different tablets or capsules, usually further comprising pharmaceutically acceptable auxiliaries and/or carriers, or in different compartments of one single capsule. Thus, in this embodiment at least the AT₁ receptor antagonist is present in a unit single dosage form physically segregated from the other active agent(s).

Examples of typical pharmaceutically acceptable auxiliaries and/or carriers for use in the formulations described above include: sugars such as lactose, sucrose, mannitol and sorbitol; starches such as cornstarch, tapioca starch and potato starch; cellulose and derivatives such as sodium carboxymethyl cellulose, ethyl cellulose and methyl cellulose; calcium phosphates such as dicalcium phosphate and tricalcium phosphate; sodium sulfate; calcium sulfate; polyvinylpyrrolidone; polyvinyl alcohol; stearic acid; alkaline earth metal stearates such as magnesium stearate and calcium stearate; stearic acid; vegetable oils such as peanut oil, cottonseed oil, sesame oil, olive oil and corn oil; non-ionic, cationic and anionic surfactants; ethylene glycol polymers; betacyclodextrin; fatty alcohols; and hydrolyzed cereal solids, as well as other non-toxic compatible fillers, binders, disintegrants, agents, e.g. talcum; buffers, preservatives, antioxidants, lubricants, flavoring and the like commonly used in pharmaceutical formulations.

In a specific embodiment of said first aspect, the invention also relates to a kit comprising in separate containers in a single package pharmaceutical dosage forms for use in combination, comprising:
i1) in one separate container a pharmaceutical dosage form comprising at least one neutral endopeptidase inhibitor and in a second separate container a pharmaceutical dosage form comprising at least one inhibitor of the endogenous endothelin producing system, or
i2) in one separate container a pharmaceutical dosage form comprising a dually acting compound capable of inhibiting neutral endopeptidase and the endogenous endothelin producing system, and
ii) in another separate container a pharmaceutical dosage form comprising at least one AT₁ receptor antagonist.

The kit form is particularly advantageous but not limited to the case when the separate components must be administered in different dosage forms or are administered at different dosage intervals. The dosage forms may desirably be oral formulations, such as tablets or capsules. The separate containers may e.g. be blister packs—in particular where the oral formulations are tablets or coated tablets, boxes or other containers commonly used to package pharmaceutical dosage forms. Preferred are alternatives of the kit which comprise in one separate container a pharmaceutical dosage form comprising a dually acting compound capable of inhibiting neutral endopeptidase and the endogenous endothelin producing system, and in another separate container a pharmaceutical dosage form comprising at least one AT₁ receptor antagonist. The kit may further comprise leaflets or other written instructions as to how the different kit constituents may best be used in order to achieve best therapeutic results with the provided combination of active ingredients.

In a second aspect, the invention also relates to a use of at least one NEP-inhibitor in combination with at least one inhibitor of the endogenous endothelin producing system and at least one AT₁ receptor antagonist, for the preparation of a pharmaceutical composition or medicament for the inhibition or treatment of a cardiovascular disease, in particular hypertension and/or cardiac insufficiency; essential hypertension and/or pulmonary hypertension in mammals and humans.

In a third aspect, the invention relates to a method of treating or inhibiting a cardiovascular disease in mammals and humans comprising administering to a subject in need thereof an effective amount of a combination of at least one NEP-inhibitor, at least one inhibitor of the endogenous endothelin producing system and at least one AT₁ receptor antagonist. Subjects in need of such treatments are in particular those humans or mammals who are suffering from or being susceptible to a cardiovascular disease, in particular hypertension and/or cardiac insufficiency; essential hypertension and/or pulmonary hypertension. Further, the combination treatment according to the present invention is also deemed suitable or beneficial for the treatment and/or inhibition of endothelial dysfunction and/or sexual dysfunction, in particular male dysfunction, more particular erectile dysfunction. The active agents (a), (b) and (c) can be obtained and administered together, sequentially one after the other or separately in one combined unit dosage form, e.g. in one tablet or capsule. Thus, the active agents can be administered in either order at the same time or at different times spread over the day, the optimal dosage regimen usually being determined by prescription of a physician.

In one specific embodiment of said third aspect, a fixed combination of a dually acting compound capable of inhibiting neutral endopeptidase and the endogenous endothelin producing system, and an AT₁ receptor antagonist can be used. Fixed combinations comprising daglutril and eprosartan, daglutril and candesartan or daglutril and losartan are preferred alternatives of this specific embodiment.

Description of the Pharmacological Test Methods

The beneficial effects of the combination therapy according to the invention can e.g. be shown in a clinical test protocol and in an animal model at the rat:

Clinical Test Protocol

A randomized, placebo-controlled, parallel group, multi-center, single dose study of oral daglutril (vide supra) during 12-hour right heart catheterization in human subjects with congestive heart failure (=CHF) was performed. Each subject received one dose of daglutril or placebo. The study consisted of three visits (or study days when in-subjects were included). Ambulatory subjects were in hospital for two nights and one day.

Criteria for evaluating efficacy were systemic vascular resistance (=SVR), pulmonary capillary wedge pressure (=PCWP), cardiac output (=CO), heart rate (=HR), pulmonary and systemic systolic, diastolic and mean pressures; pulmonary vascular resistance (=PVR); stroke volume index (=SVI); cardiac index (=Cl); transpulmonary gradient and neurohormones.

The primary efficacy parameter was the maximum decrease from baseline over 6 hours for SVR and was compared between treatment groups using analysis of covariance, with the baseline value as covariate and center and NYHA classification as factors. Testing was carried out one-sided at an overall significance level of α=0.05. Adjustment for the multiple comparisons artifact was controlled by applying Dunnett's procedure. In addition, the existence of a dose-response relationship for daglutril was evaluated by investigating linear, quadratic and cubic contrasts. The secondary efficacy parameter was the maximum change from baseline for PCWP and was analyzed in the same way as the primary variable. The maximum decrease from baseline over 12 hours, the change from for each individual time point and the adjusted area under the curve (=AUC) over 6 and 12 hours were analyzed for SVR and PCWP, using similar statistical methodology as for the main parameter of interest. All other tertiary efficacy parameters were analyzed using the same statistical methodology as for the primary efficacy parameter.

Criteria for evaluating safety were laboratory variables; electrocardiogram (=ECG); physical examinations; vital signs and adverse events (=AEs).

Criteria for inclusion comprised male or female (without childbearing potential) subjects, aged ≧18 to ≦85 years, with a history of chronic, symptomatic, mild to severe (NYHA Class II-IV) CHF for at least three months, with documented systolic dysfunction (left ventricular ejection fraction (=LVEF)≦35% by echocardiography) receiving a stable dose of their individually optimized medication regimen for at least one week prior to study enrollment.

(96) Subjects were screened and (75) were randomized and analyzed, (18) subjects in the 200 mg daglutril group, (20) subjects in the 400 mg daglutril group, (19) subjects in the 800 mg daglutril group and (18) subjects in the placebo group. In a subgroup analysis, the 75 randomized subjects in the study were divided into subgroups, namely placebo or daglutril treatment with criterion present or absent. As criterion was taken whether concomitant medication of losartan potassium was taken prior to and continued after randomization. In the placebo group 1 patient took losartan potassium whereas 15 patients did not take losartan potassium. In the daglutril group 5 patients took losartan potassium whereas 49 patients did not take losartan potassium.

Summary statistics of the average over the first 6 hours (0.5, 6 hours; only computed if no time points have missing data) (mean, Standard Deviation (=SD), n) are given. Both, for the criterion present and absent subgroups, the placebo corrected mean values and summary statistics (mean change, standard error of change (=SE) and standardized mean change (=mean/SE) are given.

In this test model, administration of daglutril in addition to a concomitant medication with an AT₁ receptor antagonist (losartan) prior to and after randomisation, respectively, showed the results on placebo corrected mean change of mean pulmonary artery pressure (=MPAP; 0.5-6 hrs) as given in the following Table 1:

TABLE 1
Pharmacological results of coadministration of daglutril and AT₁-
receptor antagonist (losartan potassium) on MPAP

| | daglutril and no | daglutril with |
|---|---|---|
| | AT₁-receptor antagonist | AT₁-receptor antagonist |
| | [mm Hg] (SE) | [mm Hg] (SE) |
| Placebo corrected | −3.35 (1.06) | −7.44 (3.45) |
| mean change of | | |
| MPAP (average | | |
| 0.5-6 hrs) | | |

The test results show that the beneficial influence on pulmonary blood pressure of a dually acting compound capable of inhibiting NEP and the endogenous endothelin producing system, namely daglutril, in addition to an AT₁-receptor antagonist was relevantly more marked than the influence that resulted from administration of a dually acting compound of inhibiting NEP and the endogenous endothelin producing system, namely daglutril, alone.

Animal Test Model

Male spontaneously hypertensive rats (=SHR, insulin resistant strain from Charles River; aged 6 months) were equipped with telemetry transmitters for continuous monitoring of blood pressure and heart rate (as described below). After 3 days of monitoring under baseline (untreated) conditions, animals were divided into two groups receiving an AT₁-receptor antagonist (eprosartan mesylate, hereinafter referred to as experiment I; or candesartan cilexetil, hereinafter referred to as experiment II) or an AT₁-receptor antagonist plus daglutril in combination. In experiment II, a third group of rats was included, receiving only daglutril. Compounds were administered via the drinking water, and daily drug intake was measured by weighing the water bottles thrice weekly. Intended daily doses in experiment I were 60 mg/kg/day of eprosartan mesylate plus, in the combination group, 100 mg/kg/day of daglutril. In experiment II, intended daily doses were 1 mg/kg of candesartan cilexetil, and 100 mg/kg of daglutril in the daglutril only and the combination group. Telemetry transmitters for continuous monitoring of blood pressure, heart rate and locomotor activity (TA11PA-C40, Data Sciences, USA) were implanted intraabdominally under inhalative halothane anesthesia. A midline abdominal incision was made, and the abdominal aorta was visualized by removal of retroperitoneal fat and connective tissue. A ligature was placed caudal of the renal arteries, the aorta was punctured with a 22G needle, and the catheter was advanced into the aorta. The entry point was sealed with tissue adhesive (Vetbond®, 3M, USA), the ligature was removed, and the abdominal incision was closed. Measurements of aortic pressure were taken every 5 minutes (=min) for 4 seconds (=s) each at a sampling rate of 500 Hz, and were corrected for the corresponding ambient pressure (ambient pressure monitor, C11PR, Data Sciences, USA).

Concentrations of AT₁-receptor antagonists and daglutril in the drinking water were adjusted once per week, in order to ensure the intended daily intake. In experiment I, the average daily water intake throughout the 33 days treatment period amounted to 51 and 56 ml/kg in the eprosartan and eprosartan plus daglutril group, respectively, resulting in the uptake of 62 mg/kg/day of eprosartan in both groups, and 104 mg/kg/day of daglutril in the combination group. In experiment II, the average daily water intake during the 25 days treatment period was 64 ml/kg (candesartan only), 62 ml/kg (daglutril only) and 62 ml/kg (candesartan plus daglutril), resulting in daily doses of 0.9 mg/kg of candesartan in both, the candesartan and combination group, and 101 mg/kg and 98 mg/kg of daglutril in the daglutril and combination group, respectively.

The blood pressure, heart rate and activity values, sampled in 5 min intervals by the Dataquest system, were used for calculation of individual 24 hours (=h)−means. These 24 h means were exported to Excel, and group mean values of systolic blood pressure (=SBP), diastolic blood pressure (=DBP), heart rate (=HR), and locomotor activity (=ACT) were calculated for the different treatment groups. For the statistical analysis, a baseline value (pre) was calculated from 3 days prior to compound application, and effects of AT₁-receptor antagonist, daglutril and their combination were calculated in relation to this baseline value (average value during the treatment period minus baseline value). The statistical comparison was done by using analysis of variance, followed by two-tailed Student's t-test for comparison of AT₁ receptor antagonist and combination groups, both at an error level of P<0.05.

In this test model, administration of daglutril, alone and in combination with an AT₁ receptor antagonist (eprosartan mesylate or candesartan cilexetil), and compared to AT₁-receptor antagonist only administration, showed the results as given in the following Tables 2 and 3:

TABLE 2
Effects of coadministration of daglutril and AT₁-receptor antagonist
(eprosartan mesylate) on cardiovascular parameters in the spontaneously
hypertensive rat

| | daglutril | | | | eprosartan + | | |
|---|---|---|---|---|---|---|---|
| CV | (1) | | Eprosartan | | daglutril | | Statistics |
| Parameters | Mean | SEM | Mean | SEM | Mean | SEM | ANOVA |
| DBP | 0.8 | 0.3 | −5.6 | 1.4 | −8.3 | 1.7 | P < 0.001 |
| [mmHg] | | | | | | | |
| SBP | −1.5 | 0.5 | −5.9 | 1.3 | −11.7* | 1.8 | P < 0.001 |
| [mmHg] | | | | | | | |
| HR [1/min] | −2.0 | 1.6 | −7.6 | 1.7 | 0.5* | 1.2 | P < 0.05 |

Shown are changes vs. matched baseline values measured before the start of treatment;
n = 5 animals per group;
SEM = Standard Error of Measurement, two-tailed ANOVA,
n.s. = not significant,
*P < 0.05 two-tailed t-test eprosartan versus eprosartan + daglutril,
(1) data from experiment II

TABLE 3
Effects of coadministration of daglutril and AT₁-receptor antagonist
(candesartan cilexetil) on cardiovascular parameters in the spontaneously
hypertensive rat

| | | | | | candesartan + | | |
|---|---|---|---|---|---|---|---|
| CV | daglutril | | Candesartan | | daglutril | | Statistics |
| Parameters | Mean | SEM | Mean | SEM | Mean | SEM | ANOVA |
| DBP | 0.8 | 0.3 | −19.7 | 2.3 | −19.9 | 1.2 | P < 0.001 |
| [mmHg] | | | | | | | |
| SBP | −1.5 | 0.5 | −22.8 | 0.7 | −28.9* | 1.7 | P < 0.001 |
| [mmHg] | | | | | | | |
| HR [1/min] | −2.0 | 1.6 | −3.0 | 1.7 | −0.2 | 3.8 | n. s. |

Shown are changes vs. matched baseline values measured before the start of treatment;
n = 5 animals per group;
two-tailed ANOVA,
n.s. = not significant,
*P < 0.01 two-tailed t-test candesartan versus candesartan + daglutril

In both experiments the decrease in systolic blood pressure was significantly greater in the combination group (t-test, P<0.05) than in the group receiving the respective AT₁-receptor antagonist alone. Moreover, daglutril, when given alone, did not lead to a reduction in blood pressure in this model.

The dosage of the active agents can depend on a variety of factors, such as mode of administration, species, age and/or individual condition. Suitable dosages for the active agents of the pharmaceutical combination according to the present invention are therapeutically effective dosages, for example those which are commercially available. Normally, in the case of oral administration, an approximate daily dose of from about 4 mg to about 600 mg is to be estimated for each of the active agents e.g. for a patient of approximately 75 kg in weight. For example, a pharmaceutical composition according to the invention may preferably comprise daglutril as dually acting compound capable of inhibiting ECE and hSEP in the range of 5-600 mg. The dose range of AT₁ receptor antagonists which are present in the pharmaceutical compositions according to the invention may vary depending on i.a. the substance used and may be (each calculated for the pure active substance, not the salt or solvate thereof), e.g., 4-32 mg for candesartan, 300-600 mg for eprosartan, 75-300 mg for irbesartan, 25-100 mg for losartan, 20-80 mg for telmisartan or 40-320 mg for valsartan. The administration of the pharmaceutical composition may occur up to three times a day. Once daily administration forms are preferred.

### EXAMPLE I

### Capsules Containing Daglutril and Losartan

Capsules with the following composition per capsule are produced:

| | |
|---|---|
| Daglutril tricalcium phosphate salt | 200 mg |
| Losartan potassium | 50 mg |
| Corn starch | 50 mg |
| Lactose | 80 mg |
| Ethyl acetate | q.s. |

The active agents, the corn starch and the lactose are processed into a homogeneous pasty mixture using ethyl acetate. The paste is ground and the resulting granules are placed on a suitable tray and dried at 45° C. in order to remove the solvent. The dried granules are passed through a crusher and mixed in a mixer with the further following auxiliaries:

| | |
|---|---|
| Talcum | 5 mg |
| Magnesium stearate | 5 mg |
| Corn starch | 9 mg |

and are then poured into 400 mg capsules (=capsule size 0).

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A pharmaceutical composition comprising pharmacologically effective amounts of each of:
a) at least one NEP-inhibitor;
b) at least one inhibitor of the endogenous endothelin producing system, and
c) at least one AT₁ receptor antagonist.

2. A pharmaceutical composition according to claim 1, further comprising at least one pharmaceutically acceptable auxiliary or carrier.

3. A pharmaceutical composition according to claim 1, further comprising acetylsalicylic acid.

4. A pharmaceutical composition according to claim 1, wherein said composition comprises an orally administrable dosage form selected from the group consisting of tablets, coated tablets, capsules, syrups, elixirs and suspensions.

5. A pharmaceutical composition according to claim 1, wherein the AT₁ receptor antagonist is present in a unit single dosage form physically segregated from the NEP-inhibitor and the inhibitor of the endogenous endothelin producing system.

6. A pharmaceutical composition according to claim 1, wherein the inhibitor of the endogenous endothelin producing system is selected from the group consisting of inhibitors of endothelin converting enzyme, inhibitors of human soluble endopeptidase, and dually acting compounds which inhibit both endothelin converting enzyme and human soluble endopeptidase.

7. A pharmaceutical composition according to claim 1, wherein the at least one neutral endopeptidase inhibitor and the at least one inhibitor of the endogenous endothelin producing system are present in the form of a dually acting compound which inhibits both neutral endopeptidase and the endogenous endothelin producing system.

8. A pharmaceutical composition according to claim 7, wherein said dually acting compound is a compound which inhibits both neutral endopeptidase and human soluble endopeptidase.

9. A pharmaceutical composition according to claim 7, wherein said dually acting compound is a compound corresponding to Forumula I 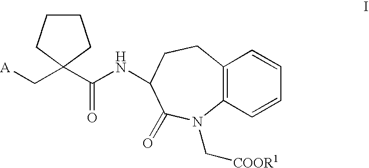
wherein
R¹ is hydrogen or a group forming a biolabile carboxylic acid ester,
A represents a group selected from the subgroups
(a) 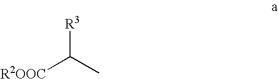
wherein
R² is hydrogen or a a group forming a biolabile carboxylic acid ester, and
R³ is a phenyl-C₁₋₄-alkyl group which can optionally be substituted in the phenyl ring by C₁₋₄-alkyl, C₁₋₄-alkoxy or halogen; or a naphthyl-C₁₋₄-alkyl group; or
(b) 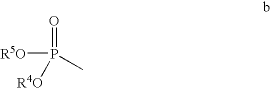
wherein
R⁴ is hydrogen or a group forming a biolabile phosphonic acid ester and
R⁵ is hydrogen or a group forming a biolabile phosphonic acid ester; and (c) 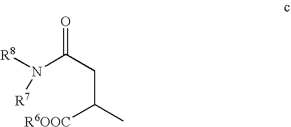
wherein
R⁶ is is hydrogen or a group forming a biolabile carboxylic acid ester,
R⁷ is hydrogen, C₁₋₄-alkyl or C₁₋₄-hydroxyalkyl, the hydroxyl group of which is optionally esterified with C₂₋₄-alkanoyl or an amino acid residue, and
R⁸ is C₁₋₄-alkyl; C₁₋₄-alkoxy-C₁₋₄-alkyl; C₁₋₄-hydroxyalkyl, which is optionally substituted by a second hydroxyl group and the hydroxyl groups of which are each optionally esterified with C₂₋₄-alkanoyl or an amino acid residue; (C₀₋₄-alkyl)₂amino-C₁₋₆-alkyl; C₃₋₇-cycloalkyl; C₃₋₇-cycloalkyl-C₁₋₄-alkyl; phenyl-C₁₋₄-alkyl, the phenyl group of which is optionally substituted 1-2 times by C₁₋₄-alkyl, C₁₋₄-alkoxy and/or halogen; naphthyl-C₁₋₄-alkyl; C₃₋₆-oxoalkyl; phenylcarbonylmethyl, the phenyl group of which is optionally substituted 1-2 times by C₁₋₄-alkyl, C₁₋₄-alkoxy and/or halogen, or 2-oxoazepanyl, or
R⁷ and R⁸ together are C₄₋₇-alkylene, the methylene groups of which are optionally replaced 1-2 times by carbonyl, nitrogen, oxygen and/or sulfur and which are optionally substituted once by hydroxy, which is optionally esterified with C₂₋₄-alkanoyl or an amino acid residue; C₁₋₄-alkyl; C₁₋₄-hydroxyalkyl, the hydroxyl group of which is optionally esterified with C₂₋₄-alkanoyl or an amino acid residue; phenyl or benzyl,
or a physiologically compatible salt thereof.

10. A pharmaceutical composition according to claim 9, wherein said dually acting compound is a compound corresponding to Formula Ia 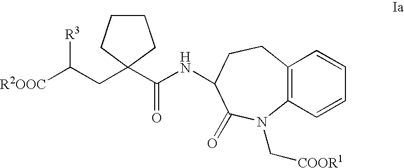
wherein
R¹ is hydrogen or a group forming a biolabile carboxylic acid ester,
R² is hydrogen or a a group forming a biolabile carboxylic acid ester, and
R³ is a phenyl-C₁₋₄-alkyl group which can optionally be substituted in the phenyl ring by C₁₋₄-alkyl, C₁₋₄-alkoxy or halogen; or a naphthyl-C₁₋₄-alkyl group;
or a physiologically compatible salt thereof.

11. A pharmaceutical composition according to claim 10, wherein said dually acting compound is selected from the group consisting of:
2-[1-(1-Carboxymethyl-2-oxo-2,3,4,5-tetrahydro-1H-benzo[b]azepin-3-ylcarbamoyl)-cyclopentylmethyl]-4-phenyl-butyric acid ethyl ester,
2-[1-(1-Carboxymethyl-2-oxo-2,3,4,5-tetrahydro-1H-benzo[b]azepin-3-ylcarbamoyl)-cyclopentylmethyl]-4-naphthalen-1-yl-butyric acid ethyl ester,
2-[1-(1-Carboxymethyl-2-oxo-2,3,4,5-tetrahydro-1H-benzo[b]azepin-3-ylcarbamoyl)-cyclopentylmethyl]-4-phenyl-butyric acid,
2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid, and
and physiologically compatible salts of any of the foregoing.

12. A pharmaceutical composition according to claim 9, wherein said dually acting compound is a compound corresponding to Formula Ic 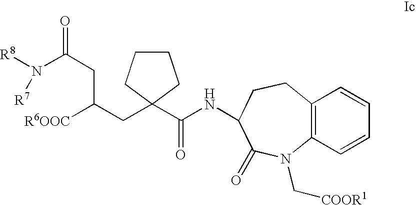
wherein
R⁶ is is hydrogen or a group forming a biolabile ester,
R⁷ is hydrogen, C₁₋₄-alkyl or C₁₋₄-hydroxyalkyl, the hydroxyl group of which is optionally esterified with C₂₋₄-alkanoyl or an amino acid residue, and
R⁸ is C₁₋₄-alkyl; C₁₋₄-alkoxy-C₁₋₄-alkyl; C₁₋₄-hydroxyalkyl, which is optionally substituted by a second hydroxyl group and the hydroxyl groups of which are each optionally esterified with C₂₋₄-alkanoyl or an amino acid residue; (C₀₋₄-alkyl)₂amino-C₁₋₆-alkyl; C₃₋₇-cycloalkyl; C₃₋₇-cycloalkyl—C₁₋₄-alkyl; phenyl-C₁₋₄-alkyl, the phenyl group of which is optionally substituted 1-2 times by C₁₋₄-alkyl, C₁₋₄-alkoxy and/or halogen; naphthyl-C₁₋₄-alkyl; C₃₋₆-oxoalkyl; phenylcarbonylmethyl, the phenyl group of which is optionally substituted 1-2 times by C₁₋₄-alkyl, C₁₋₄-alkoxy and/or halogen, or 2-oxoazepanyl, or
R⁷ and R³ together are C₄₋₇-alkylene, the methylene groups of which are optionally replaced 1-2 times by carbonyl, nitrogen, oxygen and/or sulfur and which are optionally substituted once by hydroxy, which is optionally esterified with C₂₋₄-alkanoyl or an amino acid residue; C₁₋₄-alkyl; C₁₋₄-hydroxyalkyl, the hydroxyl group of which is optionally esterified with C₂₋₄-alkanoyl or an amino acid residue; phenyl or benzyl, or
a physiologically compatible salt thereof.

13. A pharmaceutical composition according to claim 12, wherein R⁷ is hydrogen, methyl, ethyl, 2-hydroxyethyl or 3-hydroxypropyl; and each hydroxyl group is optionally esterified with C₂₋₄-alkanoyl or an amino acid residue.

14. A pharmaceutical composition according to claim 12, wherein R⁸ is isopropyl; methoxyethyl; 2-hydroxyethyl or 3-hydroxypropyl; 3-acetyloxy-n-propyl; cyclopropylmethyl; 2-methoxybenzyl, 4-methoxybenzyl; 4-methoxyphenylethyl; 2,4-dimethoxybenzyl; 1-naphthylmethyl; 3-oxo-1,1-dimethylbutyl; phenyl-2-oxoethyl; 2-(4-methoxyphenyl)-2-oxoethyl; 3-(2-oxoazepanyl); (C₀₋₄-alkyl)₂amino-C₁₋₆-alkyl, in particular dimethylamino-n-propyl, (methyl)aminoethyl, amino-n-propyl, amino-n-butyl or amino-n-pentyl, and wherein if R⁸ is 2-hydroxyethyl or 3-hydroxypropyl, each hydroxyl group optionally may be esterified with C₂₋₄-alkanoyl or an amino acid residue.

15. A pharmaceutical composition according to claim 1, wherein the AT₁ receptor antagonist is selected from the group consisting of abitesartan, benzyllosartan, candesartan, elisartan, embusartan, enoltasosartan, eprosartan, fonsartan, forasartan, glycyllosartan, irbesartan, isoteoline, losartan, milfasartan, olmesartan, opomisartan, pratosartan, ripisartan, saprisartan, saralasin, sarmesin, tasosartan, telmisartan, valsartan, zolasartan; Kissei KRH-94, Lusofarmaco LR-B/057, Lusofarmaco LR-B/081, Lusofarmaco LR B/087, Searle SC-52458, Sankyo CS-866, Takeda TAK-536, Uriach UR-7247, A-81282, A-81988, BIBR-363, BIBS39, BIBS-222, BMS-180560, BMS-184698, CGP-38560A, CGP-48369, CGP-49870, CGP-63170, C₁₋₉₉₆, CV-11194, DA-2079, DE-3489, DMP-811, DuP-167, DuP-532, GA-0056, E-4177, EMD-66397, EMD-73495, EXP-063, EXP-929, EXP-3174, EXP-6155, EXP-6803, EXP-7711, EXP-9270, FK-739, HN-65021, HR-720, ICI-D6888, ICI-D7155, ICI-D8731, KR1-1177, KT3-671, KW-3433, L-158809, L-158978, L-159282, L-159689, L-159874, L-161177, L-162154, L-162234, L-162441, L-163007, L-163017, LY-235656, LY-285434, LY-301875, LY-302289, LY-315995, ME-3221, PD-123177, PD-123319, PD-150304, RG-13647, RWJ-38970, RWJ-46458, S-8307, S-8308, SL-91.0102, U-96849, U-97018, UP-269-6, UP-275-22, WAY-126227, WK-1492.2K, WK-1360, X-6803, XH-148, XR-510, YM-358, YM-31472, ZD-6888, ZD-7155 and ZD-8731.

16. A pharmaceutical composition according to claim 1, wherein the AT₁ receptor antagonist is selected from the group consisting of abitesartan, benzyllosartan, candesartan, elisartan, embusartan, enoltasosartan, eprosartan, fonsartan, forasartan, glycyllosartan, irbesartan, losartan, milfasartan, olmesartan, opomisartan, pratosartan, ripisartan, saprisartan, tasosartan, telmisartan, valsartan, zolasartan; Kissei KRH-94, Lusofarmaco LR-B/081, Searle SC-52458, Sankyo CS-866, Takeda TAK-536, Uriach UR-7247 and physiologically compatible salts, solvates, prodrugs and esters thereof.

17. A pharmaceutical composition according to claim 1, wherein the AT1 receptor antagonist is candesartan, eprosartan or losartan.

18. A method of treating or inhibiting a cardiovascular disease in a human or other mammal patient in need thereof, said method comprising administering to said patient a pharmaceutically effective amount of a combination comprising a neutral endopeptidase inhibitor, an inhibitor of the endogenous endothelin producing system and an AT₁ receptor antagonist.

19. A method according to claim 18, wherein the cardiovascular disease is selected from the group consisting of essential hypertension, pulmonary hypertension and congestive heart failure.

20. A method according to claim 18, wherein the neutral endopeptidase inhibitor and the inhibitor of the endogenous entothelin producing system are present in the form of a dually acting compound which inhibits both neutral endopeptidase and the endogenous endothelin producing system.

21. A kit comprising in separate containers in a single package pharmaceutical dosage forms for use in combination, comprising:
i1) in one separate container a pharmaceutical dosage form comprising at least one neutral endopeptidase inhibitor and in a second separate container a pharmaceutical dosage form comprising at least one inhibitor of the endogenous endothelin producing system, or
i2) in one separate container a pharmaceutical dosage form comprising a dually acting compound which inhibits both neutral endopeptidase and the endogenous endothelin producing system; and
ii) in another separate container a pharmaceutical dosage form comprising at least one AT₁ receptor antagonist.

22. A kit according to claim 21, comprising in separate containers in a single package pharmaceutical dosage forms for use in combination, comprising:
i) in one separate container a pharmaceutical dosage form comprising a dually acting compound which inhibits both neutral endopeptidase and the endogenous endothelin producing system, and
ii) in another separate container a pharmaceutical dosage form comprising at least one AT₁ receptor antagonist.
